(19) United States
(12) Patent Application Publication
(10) Pub. No.: US 2002/0116033 A1
(43) Pub. Date: Aug. 22, 2002

(54) Controllable, wearable MRI-compatible cardiac pacemaker with pulse carrying photonic catheter and VOO functionality

(76) Inventors: Wilson Greatbatch, Akron, NY (US); Patrick Connelly, Rochester, NY (US); Michael Weiner, West Henrietta, NY (US)

Correspondence Address: Walter W. Duft, Law Office of Walter W. Duft, 10255 Main Street, Suite 10, Clarence, NY, 14031, US

(21) Appl. No.: 09/885,867

(22) Filed: Jun. 20, 2001

Related U.S. Application Data
(63) Continuation-in-part of application No. 09/864,944, filed on May 24, 2001.
(60) Provisional application No. 60/269,817, filed on Feb. 20, 2001.

(51) Int. Cl. A61N 1/362

(52) U.S. Cl. 607/33

(57) ABSTRACT

A controllable, wearable MRI-compatible, fixed-rate (VOO) pacemaker includes a self-contained power source and a pulse generator housed at the proximal end of a photonic catheter in a first enclosure designed to operate externally of a patient's body. Electrical pulses output by the pulse generator are converted into light energy and directed into the proximal end of the photonic catheter. The photonic catheter includes an optical conduction pathway over which is formed a covering of biocompatible material. Light entering the proximal end of the photonic catheter is transmitted through the optical conduction pathway, where it is collected and converted back to electrical energy at a second enclosure located at the distal end of the photonic catheter. The second enclosure houses an opto-electrical transducer that converts the optical pulses to electrical pulses and delivers them to bipolar heart electrodes. One of the electrodes may comprise the second enclosure housing the opto-electrical transducer and the other electrode can be provided by another enclosure that is spaced from the second enclosure.

### CROSS REFERENCE TO RELATED APPLICATIONS

This application is a continuation-in-part of U.S. patent application Ser. No. 09/864,944, filed on May 24, 2001, entitled “MRI-Compatible Pacemaker With Pulse Carrying Photonic Catheter Providing VOO Functionality.” This application also claims the benefit under 35 U.S.C. 119(e) of U.S. Provisional Patent Application Serial No. 60/269,817, filed on Feb. 20, 2001, entitled “Electromagnetic Interference Immune Cardiac Assist System.”

### BACKGROUND OF THE INVENTION

1. Field of the Invention

The present invention relates to wearable cardiac pacemakers. More particularly, the invention concerns a wearable cardiac pacemaker that is compatible with Magnetic Resonance Imaging (MRI). Still more particularly, the invention pertains to an MRI resistant wearable cardiac pacemaker with VOO functionality. Still more particularly, the invention involves a wearable, MRI-resistant cardiac pacemaker that is adapted to drive the heart to stress levels electrically, without drug support, while observing cardiac performance by MRI methods.

2. Description of the Prior Art

With the advent of Magnetic Resonance Imaging (MRI), it has become possible to visualize soft tissues in the human body in ways that were not previously possible. One such area is visualization of the heart itself, particularly under conditions of stress. Most contemporary temporary pacemakers have metal components, particularly lead wires, which can act as antennae in the intense MRI fields and can conduct damaging induced currents into the pacemaker structure. Also, metallic components, even if not magnetic, can shadow target areas, introducing artifacts into the MRI data.

A conventional MRI system uses three types of fields that can adversely affect pacemaker operation and cause pacemaker-induced injury to the patient. First, an intense static magnetic field, used to induce nuclear spin polarization changes in the tissue being imaged, is generated at a level of up to 1.5 Tesla (T) in clinical MRI machines and up to 6-8 T in some experimental clinical situations. Second, a time-varying gradient field, usually in the Kilohertz range, is generated for spatial encoding. Third, a Radio Frequency (RF) pulse field in a range of about 6.4-64 MHz is generated to produce an image.

These fields, acting alone or in combination with each other, can disrupt the function of the pacemaker, or possibly damage its sensitive circuits, or even destroy them. Of particular concern is the effect of induced voltages on the pacemaker's sensitive semiconductors, and magnetic field-induced activation of the reed switch that is used in the pacemaker to temporarily disable pacemaker functions for programming purposes.

Tsitlik (U.S. Pat. No. 5,217,010) attributes much of the induced voltage problem to the pacemaker electrical leads and electrodes, which together with the tissue between the electrodes, form a winding through which the MRI RF pulse field can generate substantial electromotive force. Tsitlik reports that an MRI system operating at 6.4 MHz can produce voltages of up to 20 volts peak-to-peak in this winding, and that higher frequencies produce even higher voltages. Unipolar electrode systems are said to be worse than bipolar systems. Tsitlik notes that the RF pulses propagating through the pacing leads are delivered directly to the pacemaker case itself, and that once the RF pulses are inside the case, the induced voltage can propagate along the pacemaker circuitry and cause many different types of malfunction, including inhibition or improper pacing.

A pacemaker's electrical lead system may also cause scarring of patient heart tissue during MRI scanning. This scarring is produced by necrosing currents that develop in the electrical leads as a result of large magnetic inductive forces generated by the MRI static magnetic field. If the electrical leads comprise magnetic material, they may also be mechanically displaced by the MRI magnetic field, causing additional physiological damage to the patient. Further physiological damage may result from mechanical displacement of the pacemaker case itself, which is often made of stainless steel and can be torqued or otherwise displaced by a strong magnetic field. That the power of the magnetic field generated by MRI equipment is sufficient to cause dislodgment of a pacemaker's metallic components is illustrated by one documented case in which a ferrous brain clip was fatally torn out of the brain tissue in a patient who was only in the proximity of an MRI machine.

It would be desirable to have a controllable, wearable, temporary, MRI-compatible cardiac pacemaker, which would be readily controllable by an examining physician so that he/she could easily induce cardiac stress and at the same time observe by MRI the stress effects on cardiac function. Such a device is badly needed by the medical profession, and not currently available. Temporary pacemakers exist, but all use metallic catheter leads to drive the heart. As indicated above, such leads can damage or destroy the pacemaker, and can also supply scarring burns to the cardiac wall from the MRI induced voltages in the metallic catheter.

Because of the inherent dangers of subjecting a pacemaker wearer to the strong magnetic and electromagnetic fields generated by MRI equipment, a majority of medical practitioners prohibit any type of MRI scan for such persons. Of the minority of medical practitioners who do permit MRI scans for pacemaker wearers, most will only allow scanning under limited conditions with rigid safeguards in place. Those safeguards include disabling the pacemaker while the scan is in progress, performing only emergency scans, avoiding body scans, or requiring the presence of a pacemaker expert during scanning to monitor pacemaker operation.

Accordingly, what is required is an improved controllable, wearable cardiac pacemaker that is capable of withstanding the strong magnetic and electromagnetic fields produced by MRI equipment without operational disruption and without producing physiological injury due to magnetically induced mechanical movement and electrical current. A pacemaker with this capability would allow millions of individuals who might otherwise forego potentially life-saving MRI diagnostic evaluation to receive the benefit of this important technology.

### SUMMARY OF THE INVENTION

The foregoing problems are solved and an advance in the art is provided by a controllable, wearable MRI-compatible wearable pacemaker that is characterized by a substantial absence of magnetic material and lengthy metallic lead wires, and which uses only a minimal amount of metallic material of any kind. In its most preferred embodiment, the pacemaker includes a photonic catheter having a proximal end and a distal end. A self-contained electrical power source, an electrically powered pulse generator, and an electro-optical transducer are housed at the proximal end of the photonic catheter. An opto-electrical transducer is housed at the distal end of the photonic catheter. Electrical pulse signals are delivered from the pulse generator and converted to optical pulse signals at the proximal end of the photonic catheter. The optical pulse signals are transmitted to the distal end of the photonic catheter, where they are collected and converted back to electrical pulse signals by the opto-electrical transducer. The opto-electrical transducer delivers the electrical pulse signals to a bipolar electrode pair that is also located at the distal end of the photonic catheter.

The photonic catheter of the invention can be embodied in an optical conduction pathway having a biocompatible covering. Insofar as it must be capable of transvenous insertion, the photonic catheter is preferably very small, having an outside diameter on the order of about 5 millimeters. Advantageously, because the photonic catheter is designed for optical transmission, it cannot develop magnetically-induced and RF-induced electrical currents.

The housings that contain the above-described components may be embodied in a pair of first and second enclosures, the second enclosure being a hermetically sealed non-magnetic metallic, or non-metallic, enclosure. The first enclosure houses the electrical power source, the pulse generator and the electro-optical transducer. It is adapted to be located remotely from a patient's heart and outside of the patient's body. The second enclosure houses the opto-electrical transducer. It is adapted to be implanted in close proximity to the heart and in electrical contact therewith.

Whereas the first enclosure may be of a size and shape that is consistent with conventional wearable pacemakers, the second enclosure is preferably a miniaturized housing that is generally cylindrical in shape and substantially co-equal in diameter with the photonic catheter. The second enclosure may also function as one of the pacemaker's bipolar electrodes, namely, the ring electrode. A third enclosure, mounted in closely spaced relationship to the second enclosure, but electrically insulated from it, can be used as the pacemaker's tip electrode.

The third enclosure can be constructed from the same non-magnetic metallic or non-metal material used to form the second enclosure. Because it is adapted to be inserted in a patient's heart as a tip electrode, it is generally bullet shaped. Like the second enclosure, the third enclosure preferably has an outside diameter that substantially matches the diameter of the photonic catheter. Joining the second and third enclosures is a short cylindrical span that can be made from the same material used as the optical conduction pathway's biocompatible covering. Disposed within this cylindrical span is a short length of wire that electrically connects the third enclosure to the output of the opto-electrical transducer in the second enclosure.

In the detailed description that follows, embodiments of a VOO (ventricular pacing with no feedback sensing of cardiac function) controllable, wearable cardiac pacemaker are shown and described. However, it is anticipated that the features of the invention may be used to advantage in pacemakers with other electrical configurations, such as VVI (ventricular pacing with ventricular feedback sensing and inhibited response). Similarly, it is expected that the inventive concepts described below will be applicable to other devices used for generating (or sensing) signals of biological significance in a mammalian body.

### BRIEF DESCRIPTION OF THE DRAWING

The foregoing and other features and advantages of the invention will be apparent from the following more particular description of preferred embodiments of the invention, as illustrated in the accompanying Drawing in which:

FIG. 1 is a simplified plan view of an MRI-compatible cardiac pacemaker constructed in accordance with a preferred embodiment of the invention, with an intermediate portion of the photonic catheter thereof being removed for illustrative clarity;

FIG. 2 is a partially schematic view of the pacemaker of FIG. 1, also with an intermediate portion of the photonic catheter thereof removed for illustrative clarity;

FIG. 2A is an enlarged partial perspective view of components located at the distal end of the photonic catheter portion the pacemaker of FIG. 1;

FIG. 3 is a detailed partially schematic view showing one construction of an electro-optical transducer, an opto-electrical transducer, and the photonic catheter of the FIG. 1 pacemaker, again with an intermediate portion of the photonic catheter being removed for illustrative clarity;

FIG. 4 is a schematic circuit diagram of a first exemplary pulse generator for use in the pacemaker of FIG. 1, including controls for convenient adjustment of pacemaker rate and pulse width (output energy) by an attending physician, thus permitting the creation of stress conditions in the heart without the need for drugs; and

FIG. 5 is a schematic circuit diagram of a second exemplary pulse generator similar to that of FIG. 4, but with the pulse generator incorporating a voltage doubler.

### DETAILED DESCRIPTION OF PREFERRED EMBODIMENTS

1. Overview

Applicants have determined that in order to be MRI-compatible, a wearable pacemaker should preferably have no magnetic material, no lengthy metallic lead wires, and a minimum of metallic material of any kind. These limitations have resulted in the development of an improved pacemaker that minimizes the use of electrical pathways carrying electrical signaling information to the heart. Instead, another medium is used. That medium is light. The invention advantageously provides a wearable cardiac pacemaker with VOO functionality that is largely light-driven rather than electrically-driven. As described in detail herein, this challenge is not trivial, but applicants propose solutions herein to achieve the desired goal.

2. Design Considerations

To carry light through a medium such as the human body, an optical conduction pathway is required. A glass conductor, such as glass fiber optic cable, may be used to perform this function. Glass is an excellent conductor of light and appears to offer nearly limitless information bandwidth for signals conducted over it. It transmits light over a wide spectrum of visible frequencies and beyond with very high efficiency. Glass is comprised of silicon dioxide (SiO2), as is sand and silicone rubber. However, whereas silicone rubber is readily accepted by the body, both glass and sand are summarily rejected. The reason for this is that silicone has a negative surface charge, as do blood platelets. Like charges repel and thus there is no reaction between them (assuming the absence of infection). Conversely, glass and sand both have positive surface charges. Opposite charges attract and the blood platelets are attracted to glass or sand, resulting in a foreign body reaction and sand or glass particles are rejected in a “sterile puss.” This need not be a problem because the glass fiber light pipe can be encased in a tightly bonding silicone rubber coating, or any other suitable biocompatible material, thus providing mechanical protection and a reaction-free interface in contact with the pacemaker recipient's body.

As an alternative to glass fiber, an optical conduction pathway may be implemented with plastic optical fiber, such as polyurethane or polyethylene. Although not as efficient as glass fiber, plastic fiber is ideal for short distance power and signal transmission. In a pacemaker environment, it has an additional advantage in that plastic fiber optic cable is commercially available with a polyethylene outer jacket covering. Polyethylene is a well known biocompatible material.

Glass and plastic fibers do have one problem that metal leads do not have. Namely, a glass or plastic fiber catheter would not be seen by X-ray imaging while being inserted. Thus, additional short marker metallic segments or threads may have to be included in the photonic catheter structure herein disclosed.

It will be appreciated that a pacemaker pulse generator is an electrical device and that only electrical pulses, not light, will stimulate a heart. As such, a transducer must be used to convert the pacemaker's electrical energy into light energy at the proximal end of the optical conduction pathway, and then another transducer must convert the light signal back into an electrical signal at the distal end of the optical conduction pathway. Light emitting diodes, laser diodes, and photo diodes may be used in the transducers. The preferred approach disclosed herein is to generate electrical pulses, convert the electrical pulses to optical pulses, transmit the optical pulses down a fiber optic cable, convert the optical pulses back to electrical pulses, and deliver those pulses to a pair of electrodes.

Applicants are informed that light emitting diodes, laser diodes, fiber optic light pipes, and photo diodes are all commercially available at the 20 to 200 mw level. A one millisecond electrical pulse having voltage of about 3.3 volts, a current of about three milliamperes, and a period of about 1000 milliseconds should be adequate to stimulate the heart. This represents a power level of about 10 μW (average).

3. Exemplary Pacemaker Constructions

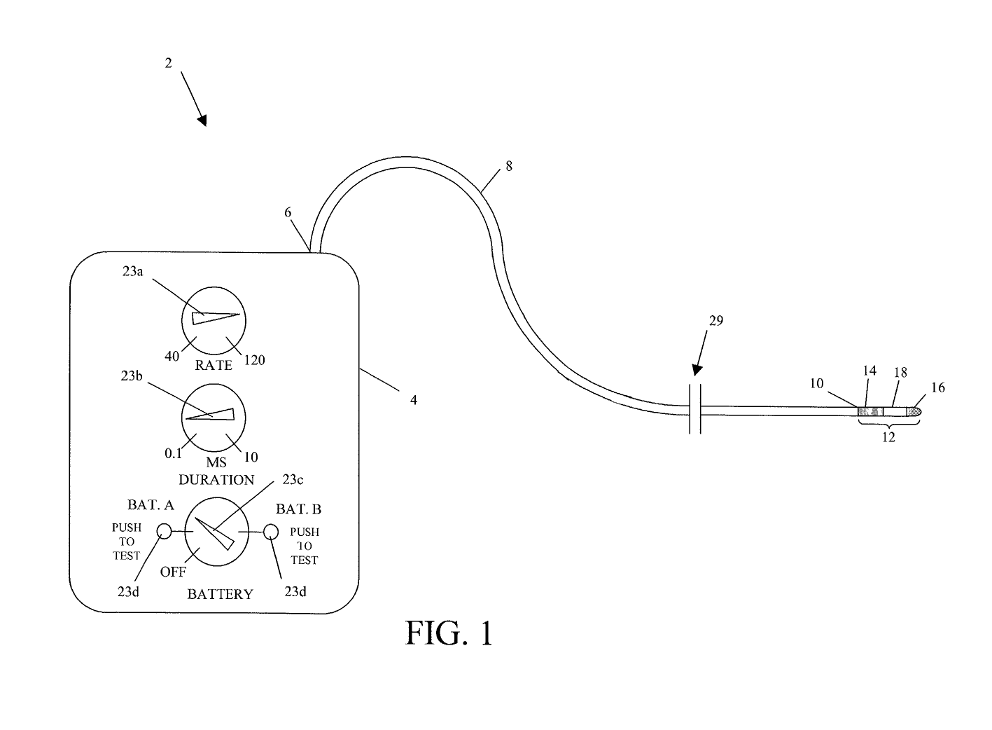

Turning now to the figures, wherein like reference numerals represent like elements in all of the several views, FIG. 1 illustrates an MRI-compatible cardiac pacemaker 2 constructed in accordance with a most preferred embodiment of the invention. The pacemaker 2 is wearable and is readily implemented to operate in a fixed-rate (VOO) mode. It includes a first (main) enclosure 4 that is designed to be located outside the body and connected to the proximal end 6 of a photonic catheter 8. A distal end 10 of the photonic catheter 8 mounts a bipolar endocardial (or pericardial) electrode pair 12 that includes a second enclosure 14 and a third enclosure 16 separated by a short insulative spacer 18. Other electrode configurations could also be used.

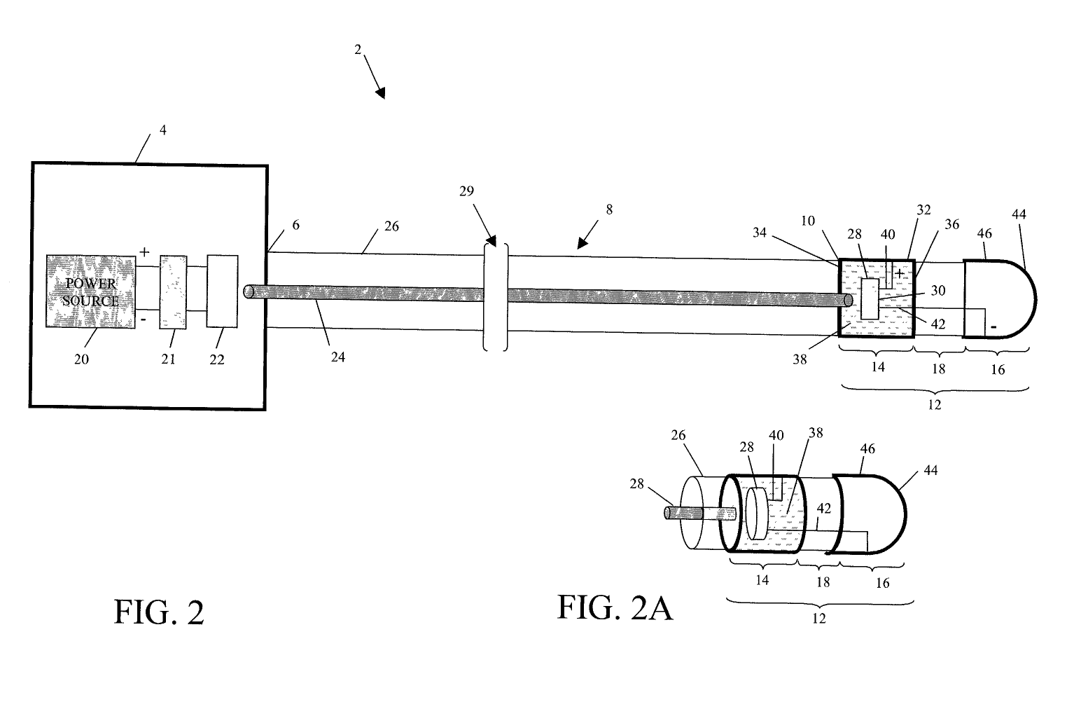

With additional reference now to FIG. 2, the main enclosure 4 houses a self-contained electrical power source 20, a pulse generator 21, and an electro-optical transducer 22. The power source 20, which may include one or more batteries, serves to deliver low energy continuous electrical power to the pulse generator. The pulse generator 21 stores the electrical energy provided by the power source 20 in one or more storage capacitors (see below), and periodically releases that energy to deliver electrical pulses to the electro-optical transducer 22. The electro-optical transducer 22 converts the electrical pulses into light energy and directs that energy into the proximal end 6 of the photonic catheter 8. The main enclosure 4 is preferably formed as a sealed casing, external to the body, made from a non-magnetic metal. The casing is of a size and shape that is consistent with conventional wearable pacemakers, and is adapted to be located remotely from a patient's heart and external to the patient's body.

Note that a rate control selector 23a and a pulse duration selector 23b can be provided on the main enclosure 4 to allow a medical practitioner to controllably stress a patient's heart by varying the rate and duration of the stimulating pulses. Note further that if the power source 20 comprises multiple batteries, these may be separately wired for independent operation and a selector switch 23c can be provided on the first enclosure 4 to selectively activate each battery for use. A pair of illuminated push buttons 23d may also be provided for testing each battery.

The photonic catheter 8 includes an optical conduction pathway 24 surrounded by a protective outer covering 26. The optical conduction pathway 24 may be constructed with one or more fiber optic transmission elements that are conventionally made from glass or plastic fiber material, e.g., a fiber optic bundle, as outlined above. As also noted above, to avoid body fluid incompatibility problems, the protective outer covering 26 should be made from a biocompatible material, such as silicone rubber, polyurethane, polyethylene, or other biocompatible polymer having the required mechanical and physiological properties. The protective outer covering 26 is thus a biocompatible covering and will be referred to as such in the ensuing discussion. Insofar as the photonic catheter 8 must be adapted for transvenous insertion, the biocompatible covering 26 is preferably a very thin-walled elongated sleeve or jacket having an outside diameter on the order of about 5 millimeters. This will render the photonic catheter 8 sufficiently slender to facilitate transvenous insertion thereof through a large vein, such as the external jugular vein.

The proximal end 6 of the photonic catheter 8 is mounted to the main enclosure 4 using an appropriate connection. The optical conduction pathway 24 may extend into the enclosure 4 for a short distance, where it terminates in adjacent relationship with the electro-optical transducer 22 in order to receive light energy therefrom. Light emitted by the electro-optical transducer 22 is directed into the proximal end 6 of the photonic catheter 8, and transmitted through the optical conduction pathway 24 to the second enclosure 14. Advantageously, because the photonic catheter 8 is designed for optical transmission, it cannot develop magnetically-induced or RF-induced electrical currents, as is the case with the metallic leads of conventional pacemaker catheters.

The second enclosure 14 houses an opto-electrical transducer 28, which converts light energy received from the distal end of the photonic catheter 8 into electrical energy. The electrical output side 30 of the opto-electrical transducer 28 delivers electrical pulses that drive the pacemaker's electrode pair 12. The second enclosure 14 is a hermetically sealed casing made from a non-magnetic metal, such as titanium, a titanium-containing alloy, platinum, a platinum-containing alloy, or any other suitable metal, including copper plated with a protective and compatible coating of the foregoing materials. Plated copper is especially suitable for the second enclosure 14 because it has a magnetic susceptibility approaching that of the human body, and will therefore minimize MRI image degradation. Note that the magnetic susceptibility of human body tissue is very low, and is sometimes diamagnetic and sometimes paramagnetic. As an alternative to using non-magnetic metals, the second enclosure 14 can be formed from an electrically conductive non-metal that preferably also has a very low magnetic susceptibility akin to that of the human body. Nonmetals that best approach this condition include conductive composite carbon and conductive polymers comprising silicone, polyethylene or polyurethane.

Unlike the main enclosure 4, the second enclosure 14 is adapted to be implanted via transvenous insertion in close proximity to the heart, and in electrical contact therewith. As such, the second enclosure 14 preferably has a miniaturized tubular profile that is substantially co-equal in diameter with the photonic catheter 8. A diameter of about 5 millimeters will be typical.

Thus, as can be seen in FIGS. 2 and 2A, the second enclosure 14 includes a cylindrical outer wall 32 and a pair of disk-shaped end walls 34 and 36. The end wall 34 is mounted to the distal end 10 of the photonic catheter 8 using an appropriate sealed connection that prevents patient body fluids from contacting the optical conduction pathway 24 and from entering the second enclosure 14. Although the photonic catheter 8 may feed directly from the main enclosure 4 to the second enclosure 14, another arrangement would be to provide an optical coupling 29 at an intermediate location on the photonic catheter. The coupling 29 could be located so that a distal portion of the photonic catheter that connects to the second enclosure 14 protrudes a few inches outside the patient's body. A proximal portion of the photonic catheter that connects to the first enclosure 14 would then be connected when MRI scanning is to be performed. Note that the first enclosure 4 could thus be located a considerable distance from the patient so as to be well outside the area of the MRI equipment, as opposed to being mounted on the patient or the patient's clothing. In an alternative arrangement, the coupling 29 could be located at the first enclosure 4.

The optical conduction pathway 24 may extend into the enclosure 14 for a short distance, where it terminates in adjacent relationship with the opto-electrical transducer 28 in order to deliver light energy thereto. Light received by the opto-electrical transducer 28 will thus be converted to electrical energy and delivered to the output side 30 of the opto-electrical transducer. Due to the miniature size of the second enclosure 14, the opto-electrical transducer 28 needs to be implemented as a miniaturized circuit. However, such components are conventionally available from commercial electronic component manufacturers. Note that the opto-electrical transducer 28 also needs to be adequately supported within the second enclosure 14. To that end, the second enclosure 14 can be filled with a support matrix material 38 that may be the same material used to form the photonic catheter's biocompatible covering 26 (e.g., silicone rubber, polyurethane, polyethylene, or any biocompatible polymer with the required mechanical and physiological properties).

As stated above, the second enclosure 14 represents part of an electrode pair 12 that delivers the electrical output of the pacemaker 2 to a patient's heart. In particular, the electrode pair 12 is a tip/ring system and the second enclosure 14 is used as an endocardial (or pericardial) ring electrode thereof. To that end, a positive output lead 40 extending from the electrical output side 30 of the opto-electrical transducer 28 is electrically connected to the cylindrical wall 32 of the second enclosure 14, as by soldering, welding or the like. A negative output lead 42 extending from the electrical output side 30 of the opto-electrical transducer 28 is fed out of the second enclosure 14 and connected to the third enclosure 16, which functions as an endocardial tip electrode of the electrode pair 12.

The third enclosure 16 can be constructed from the same non-magnetic metallic material, or non-metal material, used to form the second enclosure 14. Because it is adapted to be inserted in a patient's heart as an endocardial tip electrode, the third enclosure 16 has a generally bullet shaped tip 44 extending from a tubular base end 46. The base end 46 preferably has an outside diameter that substantially matches the diameter of the second enclosure 14 and the photonic catheter 8. Note that the base end 46 of the third enclosure 16 is open insofar as the third enclosure does not house any critical electrical components. Indeed, it mounts only the negative lead 42, which is electrically connected to the third enclosure's base end 46, as by soldering, welding or the like.

As stated above, the second enclosure 14 and the third enclosure 16 are separated by an insulative spacer 18. The spacer 18 is formed as a short cylindrical span of insulative material that may be the same material used to form the optical conduction pathway's biocompatible covering 26 (e.g., silicone rubber, polyurethane, polyethylene, or any biocompatible polymer with the required mechanical and physiological properties). Its diameter is preferably co-equal to that of the photonic catheter 8, the second enclosure 14 and the third enclosure 16. Extending through this material is the negative lead 42 that electrically connects the third enclosure 16 to the negative side of the opto-electrical transducer's output side 30. The material used to form the spacer 18 preferably fills the interior of the second enclosure 16 so that there are no voids and so that the negative lead 42 is fully captured therein. Note that the spacer 18 is mounted to the end wall 36 of the second enclosure 14 using an appropriate sealed connection that prevents patient body tissue and fluids from contacting the negative lead 42 and from entering the second enclosure 14. To connect the spacer 18 to the third enclosure 16, the latter can be press fit over the spacer, crimped thereto or otherwise secured in non-removable fashion.

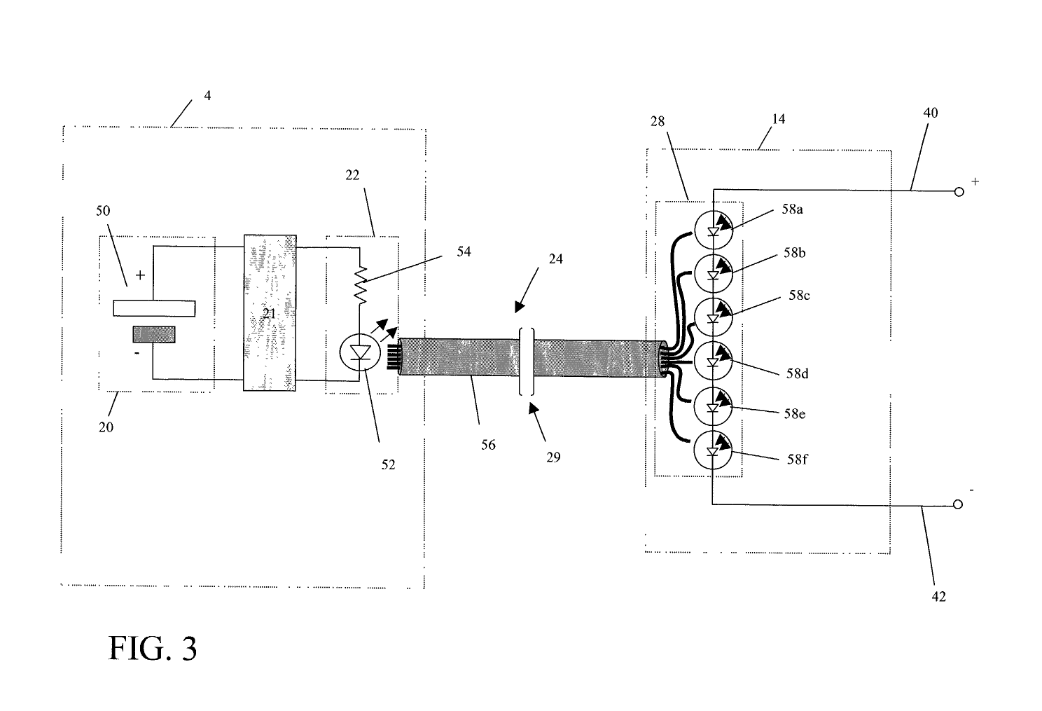

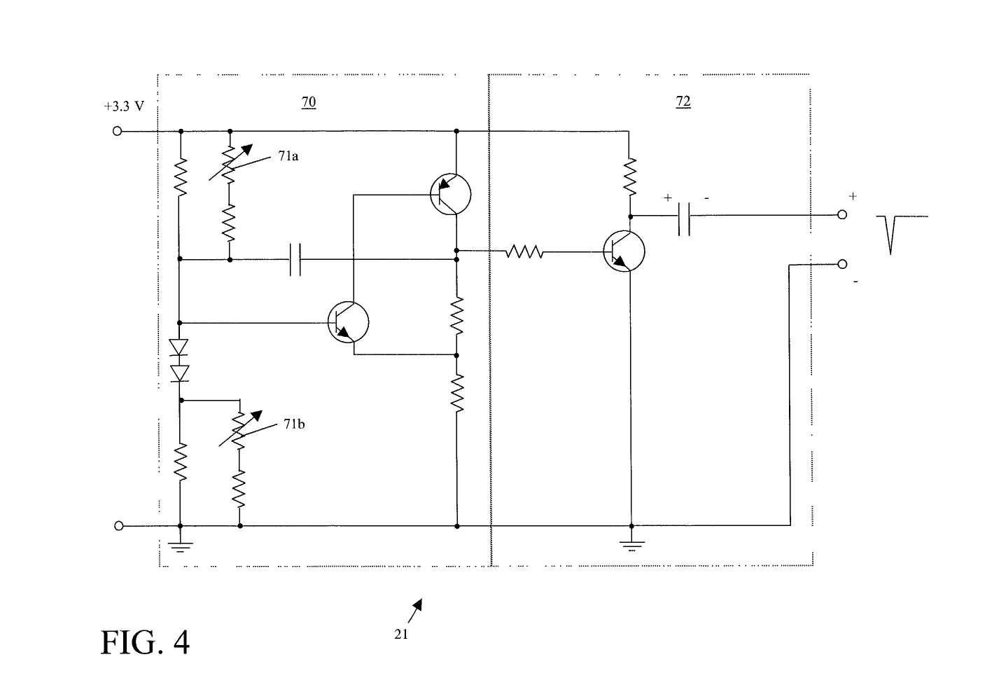

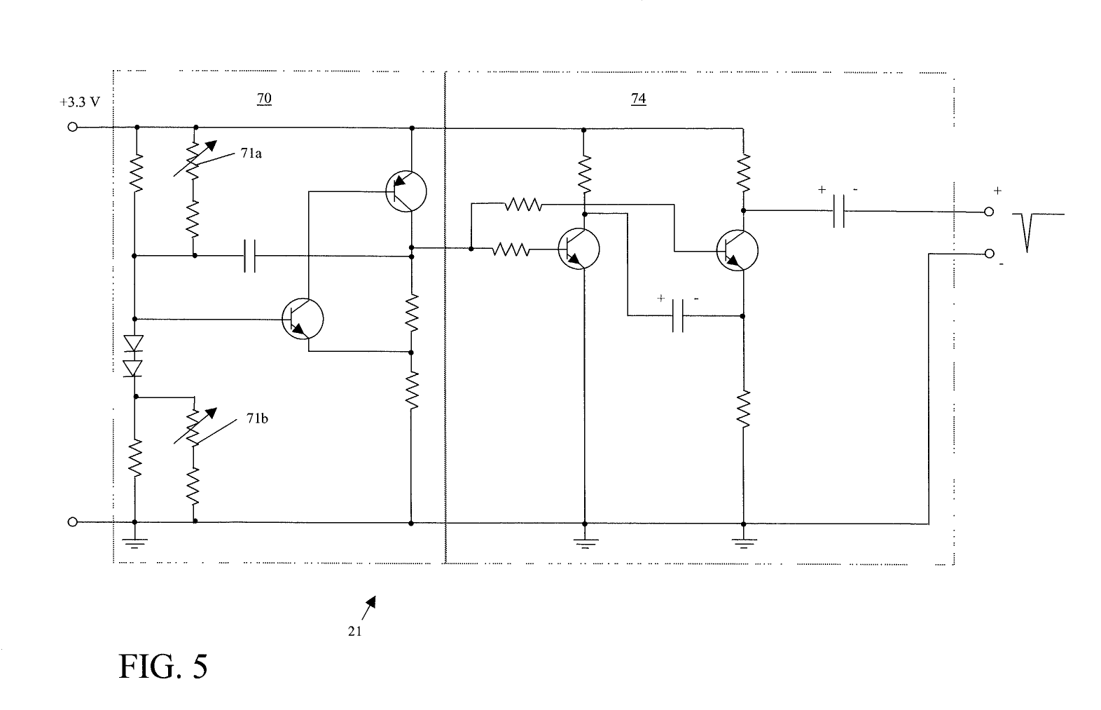

It will be appreciated that the electrical and optical components of the pacemaker 2 can be implemented in a variety of ways. By way of example, FIG. 3 shows construction details for the power source 20, the electro-optical transducer 22, the optical conduction pathway 24 and the opto-electrical transducer 28. FIGS. 4 and 5, described further below, show construction details for the pulse generator 21.

In FIG. 3, the electrical power source 20 is implemented using one or more conventional batteries 50 providing a steady state d.c. output of about 3 to 9 volts. The electro-optical transducer 22 is implemented with a light emitting or laser diode 52 and a current limiting resistor 54. The diode 52 is conventional in nature and thus has a forward voltage drop of about 2 volts and a maximum allowable current rating of about 50-100 milliamperes, or more. If additional supply voltage is available (e.g., 4 volts or higher), more than one diode 52 can be used for additional light energy output. The value of the resistor 54 is selected accordingly. By way of example, if the pulse generator 21 produces a 3 volt pulse and the desired current through a single diode 52 is 5 milliamperes, the value of the resistor 54 should be about (3-2)/0.005 or 200 ohms. This would be suitable if the diode 52 is a light emitting diode. If the diode 52 is a laser diode, other values and components would be used. For example, a current level on the order of 100 milliamperes may be required to produce coherent light output from the diode 52 if it is a laser.

The optical conduction pathway 24 in FIG. 3 can be implemented as a fiber optic bundle 56, or as a single fiber, for driving an array of photo diodes. The opto-electrical transducer 28 is implemented with six photo diodes 58a, 58b, 58c, 58d, 58e, and 58f that are wired for photovoltaic operation. The photo diodes 58a-f are suitably arranged so that each receives the light output of one or more fibers of the fiber optic bundle 56 and is forward biased into electrical conduction thereby. Each photo diode 58a-f is conventional in nature and thus produces a voltage drop of about 0.6 volts. Cumulatively, the photo diodes 58a-f develop a voltage drop of about 3.3 volts across the output leads 40 and 42 to produce a 3.3 volt pulse at a current of about 3 milliamperes across the electrode pair 12 for a total pacing power output of about 10 milliwatts. Note that the photo diodes 58a-f could be discrete devices, or they could be part of an integrated device, such as a solar cell array.

FIGS. 4 and 5 show two alternative circuit configurations that may be used to implement the pulse generator 21. Both alternatives are conventional in nature and do not constitute part of the present invention per se. They are presented herein as examples of the pulsing circuits that have been shown to function well in a pacemaker environment. In FIG. 4, the pulse generator 21 includes an oscillator 70 and an amplifier 72. The oscillator 70 is a semiconductor pulsing circuit of the type disclosed in U.S. Pat. No. 3,508,167 of Russell, Jr. (the '167 patent). As described in the '167 patent, the contents of which are incorporated herein by this reference, the pulsing circuit forming the oscillator 70 provides a pulse width and pulse period that are relatively independent of load and supply voltage. The semiconductor elements are relegated to switching functions so that timing is substantially independent of transistor gain characteristics. In particular, a shunt circuit including a pair of diodes is connected so that timing capacitor charge and discharge currents flow through circuits that do not include the base-emitter junction of a timing transistor. Further circuit details are available in the '167 patent.

Note that two additional components, variable resisters 71a and 71b, have been added to the above-described circuit to respectively provide the rate selector 23a and the pulse duration selector 23b of FIG. 1, thus allowing medical practitioners to controllably stress a patient's heart by varying the rate and duration of the stimulating pulses.

The amplifier 72 of FIG. 4 is a circuit that uses a single switching transistor and a storage capacitor to deliver a negative-going pulse of approximately 3.3 volts across the pulse generator outputs when triggered by the oscillator 70. An example of such a circuit is disclosed in U.S. Pat. No. 4,050,004 of Greatbatch (the '004 patent), which discloses voltage multipliers having multiple stages constructed using the circuit of amplifier 72. As described in the '004 patent, the contents of which are incorporated herein by this reference, the circuit forming the amplifier 72 uses a 3.3 volt input voltage to charge a capacitor between oscillator pulses. When the oscillator 70 triggers, it drives the amplifier's switching transistor into conduction, which effectively grounds the positive side of the capacitor, causing it to discharge through the pulse generator's outputs. The values of the components which make up the amplifier 72 are selected to produce an output potential of about 3.3 volts at a suitable current level to drive the one or more diodes 52. For example, if the diodes 52 are laser diodes, a current level of 100 milliamperes could be used to drive the diodes to produce optical pulses of suitable duration and period.

The amplifier 74 of FIG. 5 is a circuit that uses a pair of the amplifier circuits of FIG. 4 to provide voltage doubling action. As described in the '004 patent, the capacitors are arranged to charge up in parallel between oscillator pulses. When the oscillator 70 triggers, it drives the amplifier's switching transistors into conduction, causing the capacitors to discharge in series to provide the required voltage doubling action. The values of the components that make up the amplifier 74 are selected to produce an output potential of about 6.6 volts at a suitable current level to drive the one or more diodes 52. For example, if the diodes 52 are laser diodes, a current level of 100 milliamperes could be used to drive the diodes to produce optical pulses of suitable duration and period.

Accordingly a controllable, wearable MRI-compatible pacemaker has been disclosed that is largely light-driven rather than electrically-driven, and which is believed to offer a unique solution to the problem of MRI incompatibility found in conventional pacemakers. While various embodiments of the invention have been shown and described, it should be apparent that many variations and alternative embodiments could be implemented in accordance with the invention. For example, although the development of an MRI-compatible cardiac pacemaker is a substantial advance, it is submitted that the use of light transmission to carry signals through the human body, as disclosed herein, will have additional applications beyond the pacemaker field, perhaps as an overall replacement for signal transmission through electrical wires. Indeed, the disclosure herein of device configurations for the conduction of power and signals through mammalian body by way of light signals and photonic catheters may have significant impact on the manner in which active (self-powered) prosthetic devices are designed for wearable service. It is understood, therefore, that the invention is not to be in any way limited except in accordance with the spirit of the appended claims and their equivalents.

What is claimed is:

1. An MRI-compatible wearable cardiac pacemaker, comprising:
a photonic catheter;
a self-contained electrical power source housed at a proximal end of said photonic catheter;
electrically powered pulsing circuitry housed at said proximal end of said photonic catheter;
first power conversion means for converting the output of said pulsing circuitry to optical energy for transmission through said photonic catheter; and
second power conversion means for converting said optical energy transmitted through said photonic catheter to electrical energy;
electrodes operatively connected to said second power conversion means for receiving said electrical energy and delivering it to cardiac tissue to which said electrodes are adapted to be connected.

2. A pacemaker in accordance with claim 1, wherein said electrical power source, said pulsing circuitry, and said first power conversion means are housed in a first enclosure and said second power conversion means is housed in a second enclosure, said second enclosure being hermetically sealed and made from a material selected from the group consisting of non-magnetic materials and electrically conductive non-metal materials having very low magnetic susceptibility.

3. A pacemaker in accordance with claim 2, wherein said material comprises titanium or an alloy containing titanium.

4. A pacemaker in accordance with claim 2, wherein said material comprises platinum or an alloy containing platinum.

5. A pacemaker in accordance with claim 2, wherein said material comprises an electrically conductive composite carbon having very low magnetic susceptibility.

6. A pacemaker in accordance with claim 2, wherein said material comprises an electrically conductive polymer having very low magnetic susceptibility.

7. A pacemaker in accordance with claim 1, wherein said photonic catheter includes a fiber optic conduction pathway.

8. A pacemaker in accordance with claim 1, wherein said fiber optic conduction pathway is a glass or plastic fiber optic conduction pathway.

9. A pacemaker in accordance with claim 1 wherein said photonic catheter comprises a fiber optic conduction pathway covered by a biocompatible covering.

10. A pacemaker in accordance with claim 9 wherein said biocompatible covering comprises a material selected from the group consisting of silicone rubber, polyurethane and polyethylene.

11. A pacemaker in accordance with claim 1 further including a pacemaker tip electrode spaced from the distal end of said photonic catheter, and wherein said second power conversion means is housed in a ring electrode of said pacemaker connected to the distal end of said photonic catheter.

12. A pacemaker in accordance with claim 1 wherein tip and ring electrode are made from a material selected from the group consisting of non-magnetic and electrically conductive non-metal materials having very low magnetic susceptibility.

13. A pacemaker in accordance with claim 2 wherein said photonic catheter has an optical coupling therein that divides said photonic catheter into a proximal portion connected to said first enclosure and a distal portion connected to said second enclosure.

14. An MRI-compatible wearable cardiac pacemaker, said pacemaker comprising:
a first enclosure adapted to be located remote from a patient's heart and outside the patient's body and;
a second enclosure unit adapted to be electrically connected to the patient's heart;
an optical conduction pathway disposed between said first and second enclosures;
an optical pulse generating system in said first enclosure operatively connected to a first end of said optical conduction pathway;
an opto-electrical transducer in said second enclosure operatively connected to a second end of said optical conduction pathway;
said optical pulse generating system being adapted to provide periodic optical pulse signals through said optical conduction pathway to said opto-electrical transducer, and said opto-electrical transducer being adapted to convert said optical pulse signals into electrical pulse signals; and
electrodes operatively connected to said opto-electrical transducer for receiving said electrical pulse signals and delivering them to the patient's heart.

15. A pacemaker in accordance with claim 14 wherein said optical pulse generating system includes an electrical pulse generator and an electro-optical transducer adapted to convert the electrical signal output of said electrical pulse generating system to said optical pulse signals for placement on said optical conduction pathway.

16. A pacemaker in accordance with claim 14 wherein said opto-electrical transducer includes a photo diode circuit.

17. A pacemaker in accordance with claim 14 wherein said second enclosure comprises a hermetically sealed casing made of non-magnetic material.

18. A pacemaker in accordance with claim 17 wherein said non-magnetic material is selected from the group consisting of titanium, platinum, alloys of titanium, alloys of platinum, copper coated with a protective and compatible plating of titanium or platinum or alloys thereof, or an electrically conductive non-metal having very low magnetic susceptibility.

19. A pacemaker in accordance with claim 14 wherein said first enclosure houses a battery made from non-magnetic material, a pulse generator powered by said battery, and an electro-optical transducer electrically connected to said pulse generator and optically communicating with said first end of said optical conduction pathway.

20. A pacemaker in accordance with claim 19 wherein said electro-optical transducer comprises a light emitting diode or a laser diode.

21. A pacemaker in accordance with claim 14 wherein said optical conduction pathway comprises a fiber optic element.

22. A pacemaker in accordance with claim 21 wherein said optical conduction pathway further comprises a biocompatible covering over said fiber optic element.

23. A pacemaker in accordance with claim 22 wherein said covering comprises a jacket made from the group consisting of silicone rubber, polyurethane and polyethylene.

24. A pacemaker in accordance with claim 22 wherein said covering has an outside diameter of about 5 millimeters.

25. A pacemaker in accordance with claim 14 wherein said second enclosure comprises a hermetically sealed casing made of a material selected from the group consisting of non-magnetic metallic materials and electrically conductive non-metals having very low magnetic susceptibility.

26. A pacemaker in accordance with claim 25 wherein said non-magnetic metallic materials include materials selected from the group consisting of titanium, platinum, and alloys thereof.

27. A pacemaker in accordance with claim 25 wherein said non-metal materials include materials selected from the group consisting of electrically conductive composite carbon materials and electrically conductive polymers having very low magnetic susceptibility.

28. A pacemaker in accordance with claim 25 wherein said casing is generally cylindrical in shape.

29. A pacemaker in accordance with claim 28 wherein said optical conduction pathway is a fiber optic element having a biocompatible covering with an outside diameter, and wherein said casing has an outside diameter which is substantially coequal to said covering outside diameter.

30. A pacemaker in accordance with claim 29 wherein the outside diameter of said casing and the outside diameter of said covering are each about 5 millimeters.

31. A pacemaker in accordance with claim 30 wherein said opto-electrical transducer is carried in a matrix disposed within said casing.

32. A pacemaker in accordance with claim 31 wherein said casing functions as a ring electrode member of said electrodes.

33. A pacemaker in accordance with claim 32 further including a third enclosure adapted to be inserted in the implanted patient's heart and comprising a non-magnetic casing that electrically communicates with said opto-electrical transducer and which functions as a tip electrode member of said electrodes.

34. A pacemaker in accordance with claim 33 wherein said third enclosure is made from a material selected from the group consisting of non-magnetic metals and electrically conductive non-metals having very low magnetic susceptibility.

35. A pacemaker in accordance with claim 33 wherein said optical conduction pathway is a fiber optic element having a biocompatible covering with an outside diameter, said casings of said second and third enclosures have an outside diameter which is substantially the same as said covering outside diameter, and said second and third enclosures are separated by a cylindrical length of the material used to form said biocompatible covering.

36. A pacemaker in accordance with claim 33 wherein said optical conduction pathway, said second enclosure and said third enclosure form a catheter extending from said first enclosure, said second enclosure and said third enclosure being generally cylindrical and being joined by a generally cylindrical length of a biocompatible material to form a catheter tip, and said optical conduction pathway being a fiber optic element having a biocompatible covering with an outside diameter substantially matching that of said second and third enclosures.

37. An MRI-compatible pacemaker, comprising:
a pulse generating circuit housed in a first enclosure adapted to operate outside a patient's body and to generate periodic heart-triggering pulses;
a cardiac electrode system adapted to electrically stimulate a heart in accordance with said heart-triggering pulses; and
an optical system adapted to transport optical signals representing said heart-triggering pulses from said first enclosure to said cardiac electrode system.

38. A pacemaker in accordance with claim 37, wherein said pulse generating circuit is electrically connected to an electro-optical transducer that is co-located with said pulse generating circuit in said first enclosure, said electro-optical transducer being adapted to produce an optical pulse signal that is fed into said optical system at a power level which is in the milliwatt region.
